(19) United States
(12) Patent Application Publication
(10) Pub. No.: US 2017/0175076 A1
(43) Pub. Date: Jun. 22, 2017

(54) METHODS FOR IMPROVING PLANT EMBRYO DEVELOPMENT

(71) Applicant: Weyerhaeuser NR Company, Seattle, WA (US)

(72) Inventor: Pramod Gupta, Federal Way, WA (US)

(73) Assignee: Weyerhaeuser NR Company, Seattle, WA (US)

(21) Appl. No.: 15/345,981

(22) Filed: Nov. 8, 2016

Related U.S. Application Data
(60) Provisional application No. 62/268,671, filed on Dec. 17, 2015.

(51) Int. Cl. C12N 5/00; A01H 4/00

(52) U.S. Cl. CPC C12N 5/0025; A01H 4/006; C12N 2500/05

(57) ABSTRACT

Provided is a method of improving plant embryo development and/or germination. The method comprises developing plant embryos in the presence of nitric oxide (NO). The method entails the step of incubating plant embryogenic suspensor mass (ESM) in, or on, a development medium containing a nitric oxide donor for a period of time to develop mature somatic embryos. The method disclosed herein improves the yield and/or the germination frequency of plant embryos as compared to plant embryos developed by conventional methods without a nitric oxide donor in the medium.

### CROSS REFERENCE AND RELATED APPLICATION

This application is a nonprovisional claiming priority to U.S. Provisional Patent Application No. 62/268,671, filed Dec. 17, 2015, which is incorporated herein by reference in its entirety. To the extent the foregoing application and/or any other materials incorporated herein by reference conflict with the present disclosure, the present disclosure controls.

### FIELD OF THE TECHNOLOGY

The present technology relates to improving plant embryo development and germination.

### BACKGROUND

The demand for high quality plants, especially trees such as coniferous trees, for forestry and for making wood products continues to increase. One proposed solution to the problem of providing an adequate supply of plants is to identify individual plants that possess desirable characteristics, such as a rapid rate of growth and improved wood quality, and to reproduce these plants having the desirable traits. Thus, modern silviculture often requires the planting of large numbers of genetically identical plants that have been selected to have advantageous properties. Production of new plants by sexual reproduction, which yields botanic seeds, is usually not feasible. Asexual propagation via the culturing of somatic or zygotic embryos has been shown to yield large numbers of genetically identical embryos, each having the capacity to develop into a normal plant.

Somatic cloning is the process of creating genetically identical plants from plant somatic tissue other than the male and female gametes. In one approach to somatic cloning, plant tissue is cultured in an initiation medium which includes hormones such as auxins and/or cytokinins to initiate formation of embryogenic tissue, such as embryogenic suspensor masses, that are capable of developing into somatic embryos. Embryogenic suspensor mass (ESM) has the appearance of a whitish translucent mucilaginous mass and contains early stage embryos. The embryogenic tissue is further cultured in a multiplication medium that promotes multiplication and mass production of the embryogenic tissue. The embryogenic tissue is then cultured in a development medium that promotes development and maturation of cotyledonary somatic embryos which can, for example, be placed on a germination medium to produce germinants, and subsequently transferred to soil for further growth; or alternatively, placed within manufactured seeds and sown in the soil where they germinate to produce seedlings. Manufactured seeds are described, for example, in U.S. Pat. Nos. 5,564,224; 5,687,504; 5,701,699; and 6,119,395.

Although some plants can be reproduced by somatic cloning, somatic embryo germination sometimes is very low compared to zygotic embryo germination. Somatic embryo quality contributes to germination frequency. Therefore, there is a continuing need for methods of producing high quality plant somatic embryos to promote maturation and germination of somatic embryos.

### SUMMARY

In one aspect, a method of improving plant embryo development and/or germination is provided. The method comprises developing plant embryos in the presence of nitric oxide (NO). The method entails the step of incubating plant embryogenic suspensor mass (ESM) in, or on, a development medium containing a nitric oxide donor for a period of time to develop mature somatic embryos, wherein the yield and/or the germination frequency of mature somatic embryos are improved as compared to embryos developed by conventional methods without a nitric oxide donor in the medium. Optionally, the method includes a step of incubating the ESM in, or on, a maintenance medium for a period of time before incubating in, or on the development medium containing a nitric oxide donor. In certain embodiments, the maintenance medium can also contain a nitric oxide donor.

In certain embodiments, the plant embryos are developed in dark. In certain embodiments, the plant is a tree, such as a conifer. In certain embodiments, the nitric oxide donor is sodium nitroprusside (SNP) or nitrosyl ethylenediaminetetraacetate ruthenium (II) ([Ru(NO)(Hedta0)]).

### BRIEF DESCRIPTION OF THE DRAWINGS

FIG. 1 shows the yield of loblolly pine embryos obtained by different treatments disclosed in Example 2. The yields of all four genotypes and the average are shown in the numbers of embryos per ml of settled cells plated.

FIGS. 2A and 2B show the total germination fraction of loblolly pine embryos by different treatments. FIG. 2A shows the germination fraction of Category 1 embryos and FIG. 2B shows the germination fraction of Category 1 and Category 2 embryos.

FIG. 3 shows the effects (averaged by genotypes) of different concentrations of SNP in the development medium on the yield of Douglas-fir embryos.

FIGS. 4A and 4B show the total germination fraction of Douglas-fir embryos by different treatments. FIG. 2A shows the germination fraction of Category 1 embryos and FIG. 2B shows the germination fraction of Category 1 and Category 2 embryos.

### DETAILED DESCRIPTION

Unless specifically defined herein, all terms used herein have the same meaning as they would to one skilled in the art of the disclosed technology.

As used herein, the term “embryogenic suspensor mass” (ESM) refers to early stage embryos in the process of multiplication by budding and cleavage.

As used herein, the term “embryogenic tissue” refers to an aggregate of tens to hundreds of embryogenic cells that form an embryogenic suspensor mass.

As used herein, the term “somatic embryo” refers to an embryo produced by culturing embryogenic tissue by standard methods under laboratory conditions in which the cells comprising the tissue are separated from one another and urged to develop into minute complete embryos.

As used herein, the phrase “improving plant embryo development” means increasing the yield of plant embryos, while the phrase “improving plant embryo germination” means increasing the germination frequency of plant embryos. As disclosed herein, developing embryos in or on a medium containing a nitric oxide donor unexpectedly improves plant embryo development and plant embryo germination versus embryos developed in or on a medium without a nitric oxide donor.

As used herein, the term “germination frequency” refers to the number, proportion, percentage or fraction of germinants in a particular population of somatic embryos.

The somatic embryogenesis process is a process to develop plant embryos in vitro. This process has significant uses in forestry and in making wood products. Methods for producing plant somatic embryos are known in the art and have been previously described (see, e.g., U.S. Pat. Nos. 4,957,866; 5,034,326; 5,036,007; 5,041,382; 5,236,841; 5,294,549; 5,482,857; 5,563,061; and 5,821,126). Generally, the somatic embryogenesis process includes the steps of (1) initiating formation of embryogenic tissue (“initiation” or “induction”), such as embryogenic suspensor mass (ESM), which is a white mucilaginous mass that includes early stage embryos having a long, thin-walled suspensor associated with a small head with dense cytoplasm and large nuclei; (2) multiplying and mass producing the embryogenic tissue (“multiplication” or “maintenance”); (3) developing and forming mature cotyledonary somatic embryos (“development”); and (4) post-development processes such as stratification, singulation, and conditioning over water (COW), and then germination or placement into manufactured seeds.

Nitric oxide (NO) is an endogenous signaling molecule involved in many physiological processes in plants and acts as a mediator in the developmental and physiological processes including embryogenesis. Simontacchi et al., “Nitric oxide as a key component in hormone-regulated processes,” Plant Cell Rep. 32: 853-866 (2013). Plants can synthesize NO from nitrite by nitrate reductase (NR) or by oxidation of arginine and polyamines by enzymes. de Osti et al. reported that sodium nitroprusside (SNP) is an effect NO donor for plant cell culture and that growing plant cultures in liquid media containing SNP promotes cellular growth. de Osti et al., “Nitrosyl ethylenediaminetetraacetate ruthenium (II) complex promotes cellular growth and could be used as nitric oxide donor in plants,” Plant Science 178: 448-453 (2010). Others reported that SNP promotes multiplication and regeneration of certain plants. Han et al., “Sodium nitroprusside promotes multiplication and regeneration of Malus hupehensis in vitro plantlets,” Plant Cell Tiss. Organ. Cult. 96: 29-34 (2009). However, it has not been reported that nitric oxide acts as a starting signal in regulation of growth and morphogenesis in somatic embryogenesis of plants, particularly for conifer species.

As described herein, it has been unexpectedly discovered that providing nitric oxide to a medium by adding a nitric oxide donor, such as sodium nitroprusside or nitrosyl ethylenediaminetetraacetate ruthenium (II) ([Ru(NO)(Hedta0)]), to a standard maintenance medium and/or development medium during embryo development stage improves the yield and/or germination frequency of plant embryos as compared to the embryos developed in or on standard media without a nitric oxide donor.

The present technology provides a method of improving plant embryo development and/or germination. In several embodiments, methods in accordance with the present technology include incubating the ESM in, or on, a development medium containing a nitric oxide donor for a period of time to develop mature somatic embryos, wherein the yield and/or the germination frequency of mature somatic embryos are improved as compared to embryos developed by conventional methods without a nitric oxide donor in the medium. Optionally, the methods include incubating the ESM in, or on, a maintenance medium for a period of time before incubating in, or on the development medium containing a nitric oxide donor. The maintenance medium may or may not contain a nitric oxide donor.

The plant embryos are maintained and developed in dark for certain period of time according to standard protocols. In certain embodiments, the ESM is incubated in, or on a maintenance medium for about 3 days, about 5 days, about 1 week, or about 10 days. In certain embodiments, the ESM is incubated in, or on a development medium for about 8 weeks, about, 9 weeks, about 10 weeks, about 11 weeks, or about 12 weeks.

The nitric oxide in a medium can be provided by including a nitric oxide donor in the medium. It is within the purview of one skilled in the art to select a suitable nitric oxide donor and a suitable concentration of the nitric oxide donor. For example, nitric oxide donors that can be used include sodium nitroprusside (SNP) and nitrosyl ethylenediaminetetraacetate ruthenium (II) ([Ru(NO)(Hedta0)]). The plant ESM is maintained and/or developed in a medium containing a nitric oxide donor at a concentration ranging from about 5 μM to about 250 μM.

In certain embodiments, when a nitric oxide donor is included in a development medium, the concentration of the nitric oxide donor is from about 10 μM to about 250 μM, from about 30 μM to about 200 μM, from about 50 μM to about 150 μM, from about 75 μM to about 100 μM, about 10 μM, about 50 μM, about 75 μM, about 100 μM, about 125 μM, about 150 μM, or about 200 μM. However, it has been unexpectedly discovered that a higher concentration of nitric oxide does not necessarily result in a greater yield or a higher germination frequency.

The optimal concentration of a nitric oxide donor in the development medium may be different for different species of plants. For example, loblolly pine ESM is incubated in, or on a development medium containing a nitric oxide donor at a concentration from about 30 μM to about 250 μM, from about 50 μM to about 100 μM, about 50 μM, about 100 μM, or about 200 μM. Douglas-fir ESM is incubated in, or on a development medium containing a nitric oxide donor at a concentration from about 5 μM to about 50 μM, from about 10 μM to about 30 μM, about 5 μM, about 10 μM, or about 30 μM

In certain embodiments, the maintenance medium also contains a nitric oxide donor. The yield and/or germination frequency of the plant somatic embryos may be further increased when the maintenance medium also contains nitric oxide, provided by a nitric oxide donor. In some embodiments, the maintenance medium contains a nitric oxide donor in a concentration lower than the concentration of the nitric oxide donor in the development medium. For example, the concentration of a nitric oxide donor in a maintenance medium is about 1 μM, about 2 μM, about 3 μM, about 4 μM, about 5 μM, about 6 μM, about 7 μM, about 8 μM, about 9 μM, or about 10 μM.

Surprisingly, not only the yield but also the germination frequency of plant embryos is increased when embryos are developed in the presence of nitric oxide. Nitric oxide is not needed during germination stage to achieve the improvement described in this disclosure.

In some embodiments, the nitric oxide donor in the maintenance medium is the same as the nitric oxide donor in the development medium. In other embodiments, the nitric oxide donor in the maintenance medium is different from the nitric oxide donor in the development medium.

The methods disclosed herein can be applied to develop different plant somatic embryos, for example, somatic embryos of trees, such as conifers. In some embodiments, the conifer is loblolly pine or Douglas-fir. In certain embodiments, the maintenance medium or the development medium used in this technology is a liquid medium, a semi-solid medium, or a solid medium.

After the development period, the somatic embryos can optionally be transferred to a maturation medium, and then subjected to post-development steps such as singulation, stratification, germination, placement into manufactured seeds, and transferring to soil for further growth and development.

As shown in the working examples, the presence of nitric oxide in the development medium and/or the maintenance medium increased the yield of plant embryos by about 5%, about 10%, about 15%, about 20%, about 25%, about 30%, about 35%, about 40%, about 45%, about 50%, about 60%, about 70%, or about 80% as compared to the yield of embryos developed by conventional methods without nitric oxide. Additionally or alternatively, the presence of nitric oxide in the development medium and/or the maintenance medium increased the germination frequency of plant embryos by about 5%, about 10%, about 15%, about 20%, about 25%, about 30%, about 35%, about 40%, about 45%, about 50%, about 60%, about 70%, or about 80% as compared to the germination frequency of embryos developed by conventional methods without nitric oxide.

The following examples are provided for the purpose of illustration and are not intended to limit the disclosure in any way.

### EXAMPLES

### Example 1

Prior patents, for example, U.S. Pat. Nos. 5,036,007; 5,563,061; and 8,999,713, disclose culturing conifers. The contents of these patents are incorporated herein by reference in their entireties. This example shows the compositions of the media used in the examples that follow.

TABLE 1
Douglas-fir Basic Culture Media (BM)

| | |
|---|---|
| Constituent | mg/L |

Basal Salts

| | |
|---|---|
| KNO₃ | varies |
| CaCl₂•6H₂O | 200.0 |
| Ca(NO₃)₂•2H₂O | varies |
| KH₂PO₄ | 340.0 |
| MgSO₄•7H₂O | 400.0 |
| MnSO₄•H₂O | 20.8 |
| ZnSO₄•7H₂O | 8.0 |
| CuSO₄•5H₂O | 0.024 |
| FeSO₄•7H₂O | 27.85 |
| Na₂EDTA | 37.25 |
| H₃BO₃ | 5.0 |
| NaMoO₄•2H₂O | 0.20 |
| CoCl₂•6H₂O | 0.025 |
| KI | 1.00 |

Organic Additives

| | |
|---|---|
| Myo-Inositol | varies |
| Thiamine•HCl | 1.00 |
| Nicotinic acid | 0.50 |
| Pyridoxine•HCl | 0.50 |
| Glycine | 2.00 |
| L-Glutamine | varies |
| Casamino acids | 500.0 |
| Sugar | varies |
| pH | 5.7 |

TABLE 2
Formulations of Douglas-fir Media

| | BM-2 | BM-3 | BM-4 | BM-5 |
|---|---|---|---|---|
| | Mainte- | Singu- | Devel- | Germi- |
| | nance | lation | opment | nation |
| KNO₃ | 1250 | 1050 | 2500 | 1170 |
| Ca(NO₃)₂••2H₂O | — | 200 | — | 220 |
| Myo-Inositol | 5000 | 100 | 100 | 100 |
| L-Glutamine | 1000 | 1000 | 750 | — |
| Amino | — | — | 290 | — |
| acid mixture⁽²⁾ | | | | |
| Maltose | 30,000 | 20,000 | 25000 | — |
| Sucrose | | | | 20000 |
| PEG 8000 | — | — | 190,000 | — |
| 2.4-D | 1.1 | — | — | — |
| N6-Benzyladenine | 0.22 | — | — | — |
| Kinetin | 0.22 | — | — | — |
| Abscisic acid | — | 10/5/5 | 10 | — |
| Gibberellin 4/7 | | | 7.5 | |
| Activated charcoal | — | — | 1000 | 2500 |
| Agar | 5000 | — | — | 8000⁽³⁾ |
| Gelrite | — | — | 3000 | — |

⁽¹⁾All units are in mg/L (or ppm).
⁽²⁾L-Proline - 100, L-Asparagine - 100, L-Arginine - 50, L-Alanine - 20, L-Serine - 20.
⁽³⁾Tissue culture agar.

The pH of all media are adjusted to 5.7.
Stratification medium BM-5 without Agar.
GELRITE: gellan gum (an agar substitute);
2,4-D: 2,4-dichlorophenoxyacetic acid;
Kinetin: 6-furfurylaminopurine.

TABLE 3
Formulations of Loblolly Pine Media

| | | | | |
|---|---|---|---|---|
| BM 2 | | BM 4 | BM 5 | BM 6 |
| Mainte- | BM 3 | Devel- | Strati- | Germi- |
| nance | Rinse | opment | fication | nation |

Salts (mg/L)

| | | | | | |
|---|---|---|---|---|---|
| NH₄NO₃ | 150 | 150 | 150 | 150 | 206.25 |
| KNO₃ | 909.9 | 909.9 | 909.9 | 909.9 | 1170 |
| Ca(NO₃)₂•4H₂O | 236.15 | 236.15 | 236.15 | 236.15 | |
| MgSO₄•7H₂O | 246.5 | 246.5 | 246.5 | 246.5 | 185 |
| Mg(NO₃)₂•6H₂O | 256.5 | 256.5 | 256.5 | 256.5 | |
| MgCl₂•6H₂O | 50 | 50 | 50 | 50 | |
| KH₂PO₄ | 136 | 136 | 136 | 136 | 85 |
| CaCl₂•2H₂O | 50 | 50 | 50 | 50 | 220 |
| KI | 4.15 | 4.15 | 4.15 | 4.15 | 0.415 |
| H₃BO₃ | 15.5 | 15.5 | 15.5 | 15.5 | 3.1 |
| MnSO₄•H₂O | 10.5 | 10.5 | 10.5 | 10.5 | 8.45 |
| ZnSO₄•7H₂O | 14.4 | 14.4 | 14.4 | 14.4 | 4.3 |
| Na₂MoO₄•2H₂O | 0.125 | 0.125 | 0.125 | 0.125 | 0.125 |
| CuSO₄•5H₂O | 0.125 | 0.125 | 0.125 | 0.125 | 0.0125 |
| CoCl₂•6H₂O | 0.125 | 0.125 | 0.125 | 0.125 | 0.0125 |
| FeSO₄•7H₂O | 27.87 | 27.87 | 27.87 | 13.93 | 13.93 |
| Na₂EDTA | 37.26 | 37.26 | 37.26 | 18.63 | 18.63 |

Vitamins/Amino Acids (mg/L)

| | | | | | |
|---|---|---|---|---|---|
| Nicotinic Acid | 0.5 | 0.5 | 0.5 | 0.5 | 0.5 |
| Pyridoxine HCl | 0.5 | 0.5 | 0.5 | 0.5 | 0.5 |
| Thiamine HCl | 1 | 1 | 1 | 1 | 1 |
| Glycine | 2 | 2 | 2 | 2 | 2 |
| L-proline | | 100 | 100 | 100 | |
| L-asparagine | | 100 | 100 | 100 | |
| L-arginine | | 50 | 50 | 50 | |
| L-alanine | | 20 | 20 | 20 | |
| L-serine | | 20 | 20 | 20 | |
| PEG 8000 mw | | | 10000 | | |

Sugar/Agar mg/L

| | | | | | |
|---|---|---|---|---|---|
| Myo-Inositol | 200 | 100 | 100 | 100 | 100 |
| Casein | 500 | 500 | 500 | 500 | |
| hydrolysate | | | | | |
| L-glutamine | 1000 | 1000 | 1000 | 1000 | |
| Sucrose | | | | 25000 | 20000 |
| Maltose | 30000 | 25000 | 25000 | | |
| Glucose | | | 10000 | | |
| GELRITE | | | 2500 | 2500 | |
| TC Agar | | | | | 8000 |
| Activated carbon | | | 1000 | 1000 | 2500 |

Hormones mg/L

| | | | |
|---|---|---|---|
| ABA | | 0 | 25 |
| 2,4-D | 1.1 | | |
| BAP | 0.63 | 0.1 | |
| Kinetin | 0.61 | 0.1 | |
| epi-brassinolide | 0.96 | | |

GELRITE: gellan gum (an agar substitute);
ABA: abscisic acid
2,4-D: 2,4-dichlorophenoxyacetic acid;
BAP: 6-benzylaminopurine;
Kinetin: 6-furfurylaminopurine.

### Example 2

This example shows the effects of increased concentrations of nitric oxide on somatic embryo development and germination of loblolly pine.

### Materials and Method

Loblolly pine embryogenic suspensor mass (ESM) from four different genotypes were maintained in a standard liquid maintenance medium with 5 μM SNP or without SNP (control) at room temperature in dark for one week. One mL of 1:1 mix of settled cells and rinse medium of each genotype was plated with Decotex 2″×2″ membranes to standard semi-solid development media containing various concentrations of SNP, 50 μM, 200 μM and 500 μM. A standard development medium containing no SNP was used as a control. The plating procedure was performed as described in U.S. Pat. No. 8,925,245, the content of which is incorporated by reference in its entirety. Each plating consisted of 6 plates of each genotype. Development was carried out for 12 weeks at room temperature in dark. The plates were visually assessed at 9 weeks.

Symmetrical embryos without obvious defects that had four or more cotyledons without any fused cots or cots sprouting from the center were chosen. Embryos selected had all three parts, cotyledons, hypocotyl, and radicle regions although the sizes of embryos varied. A slight curve to the hypocotyl region was acceptable. Embryos with split radicle regions were avoided. Embryos chosen were opaque, and acceptable colors were shades of white, yellow or green. No translucent embryos or vitrified green embryos were chosen. The good quality embryos are classified in two categories: Category 1 embryos have developed root (about 1 mm), hypocotyl, epicotyl (about 5 mm) and at least 5 leaves; and Category 2 embryos have developed root, hypocotyl and epicotyl.

Post-development, the loblolly pine somatic embryos developed with various concentrations of SNP were subjected to stratification on BM-5 medium for 4 weeks at 2-4° C. in dark, followed by conditioning over water (COW) for two weeks. Subsequently, the somatic embryos were hand transferred for germination using the method as described in U.S. Pat. No. 9,090,872, the content of which is incorporated by reference herein in its entirety.

### Results

Visual Inspection Results

Volume of SCV (settled cell volume) was recorded for each flask and is shown in Table 4 below. Although the treatment with SNP in the maintenance media slightly improved by visual inspection in three out of four genotypes compared to control, there was no clear difference in settled cell volume.

TABLE 4
SCV growth after one week in maintenance media

| | Mainte- | Initial | Final | |
|---|---|---|---|---|
| | nance | Volume | Volume | SCV |
| Genotype | Medium | (mL) | (mL) | Appearance |
| Genotype A | Control (no SNP) | 50 | 120 | Looks good |
| | 5 μM SNP | 50 | 120 | Looks a little better |
| | | | | than control |
| Genotype B | Control (no SNP) | 50 | 59 | Stressed |
| | 5 μM SNP | 50 | 85 | Less stressed |
| Genotype C | Control (no SNP) | 50 | 60 | Similar to treatment |
| | 5 μM SNP | 50 | 61 | Similar to control |
| Genotype D | Control (no SNP) | 50 | 70 | Calloused |
| | 5 μM SNP | 50 | 57 | Less calloused |
| | | | | than control |

Eight treatments with various concentrations of SNP in maintenance media and/or development media were used in this experiment, detailed in Table 5 below.

TABLE 5
SNP Concentrations in Different Treatments

| | SNP Concentration | SNP Concentration |
|---|---|---|
| | in Maintenance | in Development |
| Treatment | Medium (μM) | Medium (μM) |
| 1 | 0 | 0 |
| 2 | 0 | 50 |
| 3 | 0 | 200 |
| 4 | 0 | 500 |
| 5 | 5 | 0 |
| 6 | 5 | 50 |
| 7 | 5 | 200 |
| 8 | 5 | 500 |

At the end of development, it was observed that treatments with SNP in development media had reduced extraneous ESM than the controls. The effect of reduced extraneous ESM increased with higher concentration of SNP in the development medium.

Yield

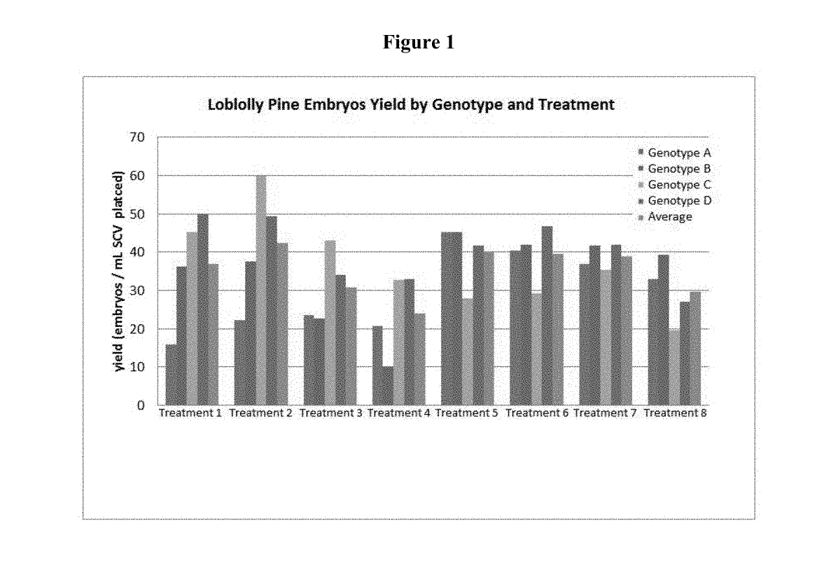

As shown in FIG. 1, all four genotypes were growing, and all appeared to be responding the same way. Treatment 2 (no SNP in maintenance medium and 50 μM SNP in development medium) and Treatment 6 (5 μM SNP in maintenance medium and 50 μM SNP in development medium) appeared to have the highest yield of good quality embryos. Treatment 4 (no SNP in maintenance medium and 500 μM SNP in development medium) and Treatment 8 (5 μM SNP in maintenance medium and 500 μM SNP in development medium) appeared to have decreased yield than the controls. Overall, Treatments 5-8 which were maintained in maintenance media containing 5 μM SNP produced higher yield than Treatments 1-4 which were maintained in maintenance media containing no SNP. Additionally, Treatments 5-8 appeared to have less ESM than Treatments 1-4.

These visual observations suggest that SNP in maintenance media reduces ESM and improves embryo quality, and that SNP in development media improves embryo quality up to a concentration of 200 μM but not at higher concentration, such as at 500 μM.

Germination

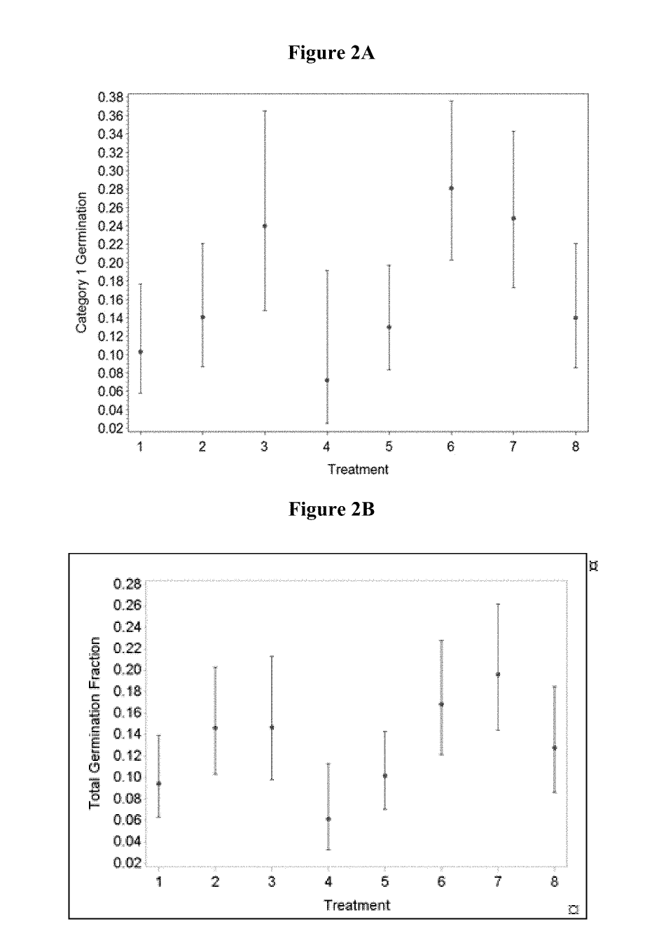

Good quality germinants having all three parts, root, hypocotyl and epicotyl, visible were selected and plotted. FIGS. 2A and 2B show the statistical analysis of total germination fraction. There is significant evidence of an overall treatment effect (p-value=0.0137). There is moderate evidence of a maintenance effect (p-value=0.0458).

All genotypes with embryos developed in the presence of SNP demonstrated improved germination percentage compared to the control with embryos developed in the absence of SNP: Genotype A improved by 18%, Genotype B improved by 39%, Genotype C improved by 16%, and Genotype D improved by 5%.

Adding 5 μM SNP to maintenance media resulted in a mild but statistically significant increase in germination percentage. Adding 50 μM to 200 μM SNP to development media resulted in a stronger and statistically significant increase in germination percentage. However, adding 500 μM SNP to development media did not result in any significant difference in germination relative to control. The root length of the germinants did not show improvements at the various SNP concentrations tested.

### Example 3

This example shows the effects of increased concentrations of nitric oxide on somatic embryo development and germination of Douglas-fir. Unless specified below, the procedure is similar to Example 2.

### Materials and Method

Douglas-fir embryogenic suspensor mass (ESM) from five different genotypes were maintained in a standard liquid maintenance medium, BM-2 medium, at room temperature in dark for one week. After singulation, all cultures were grown in a singulation medium, BM-3 medium, through ABA I, II and III stages. 3 mL of settled cells of each genotype was plated with Decotex 2″×2″ membranes to standard semi-solid development media (BM-4) containing various concentrations of SNP, 10 μM, 50 μM, 100 μM and 200 μM. A standard development medium (BM-4) containing no SNP was used as a control. The plating procedure was performed as described in U.S. Pat. No. 8,925,245, the content of which is incorporated by reference in its entirety. Each plating consisted of 6 plates of each genotype. Development was carried out for 10 weeks at room temperature in dark. The plates were visually assessed at 9 weeks.

After completion of 10 weeks incubation on development medium, cultures were transferred to a standard germination medium, BM-5 medium, for stratification treatment in dark at 2-4° C. for 4 weeks. Embryos were then sorted and kept for germination on the standard germination medium for 8 weeks at room temperature with light. Good quality germinants having all three parts, root, hypocotyl and epicotyl, visible were selected and plotted.

### Results

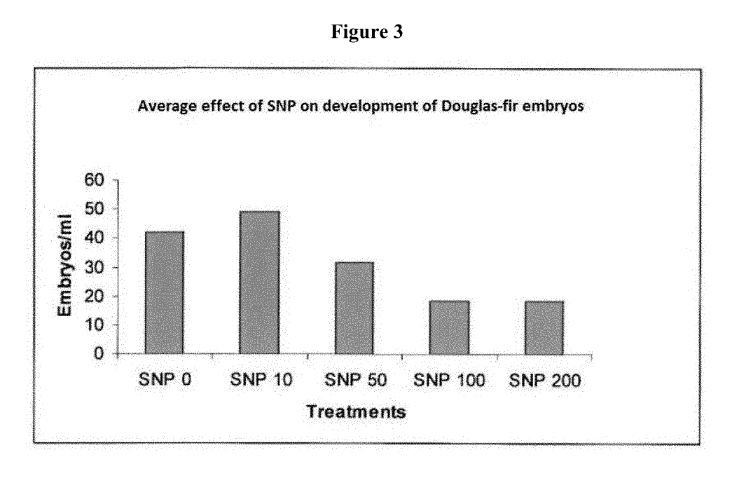

Effects of different concentrations of SNP in the development medium on Douglas-fir embryo development are shown in Table 6 below and in FIG. 3.

TABLE 6
Effects of SNP on Douglas-fir embryo
development (embryos per ml)

| | SNP | SNP | SNP | SNP | SNP |
|---|---|---|---|---|---|
| Genotype | (0 μM) | (10 μM) | (50 μM) | (100 μM) | (200 μM) |
| A | 20 | 30 | 25 | 10 | 22 |
| B | 101 | 91 | 51 | 26 | 32 |
| C | 40 | 42 | 15 | 18 | 14 |
| D | 30 | 60 | 47 | 28 | 11 |
| E | 20 | 23 | 20 | 9 | 12 |
| Average | 42.2 | 49.2 | 31.6 | 18.2 | 18.2 |

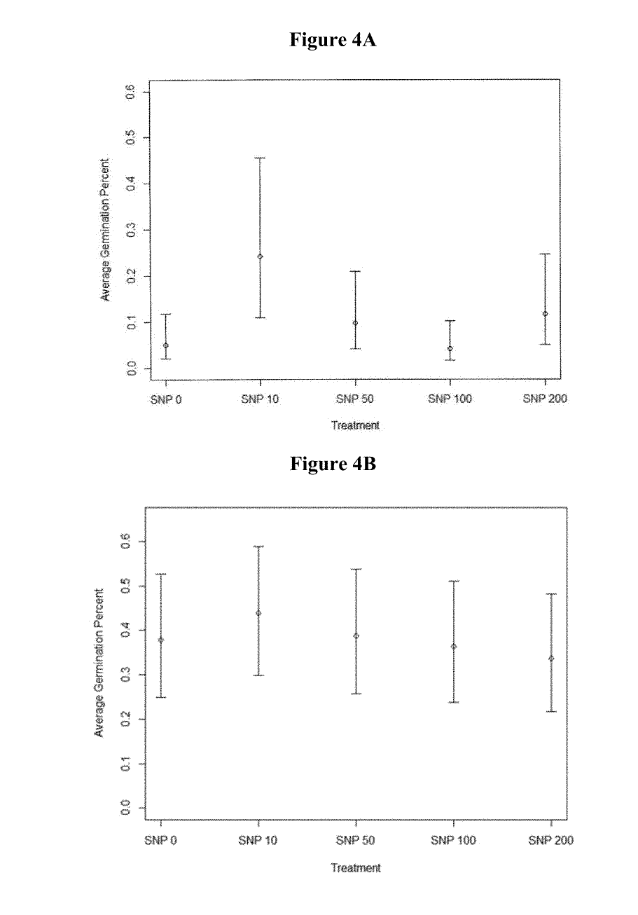

The germination percentage of all genotypes is shown in Table 7 and FIGS. 4A and 4B. Lower concentration of SNP at 10 μM demonstrated improvement in Douglas-fir embryo yield and germination frequency. However, higher concentrations of SNP did not improve development and germination of Douglas-fir embryos.

TABLE 7
Effects of SNP on Douglas-fir embryo germination percentage

| | SNP | SNP | SNP | SNP | SNP |
|---|---|---|---|---|---|
| Genotype | (0 μM) | (10 μM) | (50 μM) | (100 μM) | (200 μM) |
| A | 37.3 | 55.4 | 51.7 | 54.2 | 54 |
| B | 60.9 | 66.3 | 57.4 | 48.4 | 51.5 |
| C | 42 | 46.7 | 27 | 21.4 | 25.8 |
| D | 22.5 | 27.5 | 19.2 | 23.8 | 13.1 |
| E | 32.5 | 26.1 | 42 | 37 | 30 |
| Average | 39.04 | 44.4 | 39.46 | 36.96 | 34.88 |

We claim:

1. A method of improving plant embryo development and/or germination comprising incubating plant embryogenic suspensor mass (ESM) in, or on, a development medium comprising a nitric oxide donor for a period of time to develop mature somatic embryos, wherein the yield and/or the germination frequency of mature somatic embryos are improved as compared to embryos developed by conventional methods without a nitric oxide donor in the medium.

2. The method of claim 1, further comprising incubating the ESM in, or on, a maintenance medium for a period of time before incubating the ESM in, or on, the development medium comprising the nitric oxide donor.

3. The method of claim 2, wherein the maintenance medium comprises a nitric oxide donor.

4. The method of claim 2, wherein the ESM is incubated in, or on, the maintenance medium in dark.

5. The method of claim 1, wherein the ESM is incubated in, or on, the development medium in dark.

6. The method of claim 1, wherein the plant is a tree.

7. The method of claim 1, wherein the plant is a conifer.

8. The method of claim 1, wherein the nitric oxide donor is sodium nitroprusside (SNP).

9. The method of claim 1, wherein the nitric oxide donor is nitrosyl ethylenediaminetetraacetate ruthenium (II) ([Ru(NO)(Hedta0)]).
